(19) United States
(12) Patent Application Publication
(10) Pub. No.: US 2011/0280939 A1
(43) Pub. Date: Nov. 17, 2011

(54) ORAL PARTICLE INCLUDING PSEUDOEPHEDRINE HYDROCHLORIDE AND CETIRIZINE DIHYDROCHLORIDE

(75) Inventor: TA-PING LIAO, CHIA YI CITY (TW)

(73) Assignee: EVEREST PHARM. INDUSTRIAL CO., LTD., CHIA YI HSIEN (TW)

(21) Appl. No.: 13/176,246

(22) Filed: Jul. 5, 2011

Related U.S. Application Data
(62) Division of application No. 12/003,687, filed on Dec. 31, 2007, now abandoned.

(51) Int. Cl. A61K 9/14; A61K 31/495; A61P 37/08; A61K 9/62

(52) U.S. Cl. 424/458; 424/495; 514/255.04

(57) ABSTRACT

The present invention relates to an oral particle including pseudoephedrine hydrochloride and cetirizine dihydrochloride, which primarily includes a nucleus having a diameter ranging 25˜40 mesh, a pseudoephedrine-hydrochloride layer coated outside the nucleus with a coating solution composed of pseudoephedrine hydrochloride, a binder, a lubricant and pure water/alcohol, a release-control layer coated outside the pseudoephedrine-hydrochloride layer, and a cetirizine-dihydrochloride layer coated outside the release-control layer with a coating solution composed of cetirizine dihydrochloride, a binder, a lubricant and pure water/alcohol. Accordingly, by distributing pseudoephedrine hydrochloride and cetirizine dihydrochloride into hundreds of the particles and controlling the dissolution rate with the release-control layer, the particles can perform good absorption efficiency, and quick, stable and long-term edicinal effect.

### RELATED APPLICATIONS

This application is a Divisional patent application of co-pending application Ser. No. 12/003,687, filed on 31 Dec. 2007. The entire disclosure of the prior application, Ser. No. 12/003,687, from which an oath or declaration is supplied, is considered a part of the disclosure of the accompanying Divisional application and is hereby incorporated by reference.

### BACKGROUND OF THE INVENTION

1. Field of the Invention

The present invention relates to an oral particle including pseudoephedrine hydrochloride and cetirizine dihydrochloride, and particularly to an oral particle performs good absorption efficiency, quick and stable response effect and long-term medicinal effect by distributing pseudoephedrine hydrochloride and cetirizine dihydrochloride among hundreds of the particles and controlling the dissolution rate with the release-control layer.

2. Related Prior Arts

The oral medicine including pseudoephedrine hydrochloride and cetirizine dihydrochloride is generally used to treat Seasonal and perennial allergic rhinitis, for example, nasal congestion, sneeze, runny nose and nose and eyes itch. Currently, the market oral medicine including pseudoephedrine hydrochloride and cetirizine dihydrochloride is in the form of a tablet, for example, Zytec®, which primarily contains a pseudoephedrine-hydrochloride layer and a thin cetirizine-dihydrochloride layer outside the pseudoephedrine-hydrochloride layer. For such a tablet, enough dosage of pseudoephedrine hydrochloride and cetirizine dihydrochloride is required to achieve a therapeutic effect, which therefore results to a larger volume.

When one takes the tablet including pseudoephedrine hydrochloride and cetirizine dihydrochloride, bioavailability is limited and absorption is slow as the single tablet has only limited surface area for acting with gastric acid. In addition, the cetirizine-dihydrochloride layer coated outside of the pseudoephedrine-hydrochloride layer is hydrophilic and will be completely dissolved while being taken and only pseudoephedrine hydrochloride is remained to act with gastric acid to form adhesive colloid for slow release. Due to the small superficial measure thereof, the rate of dissolution can vary depending on the different human bodies to cause the fact that the tablet can not last and stable the medicine effect and further influence the therapeutic effect that the tablet is applied for the aforesaid diseases. As a result, people take a big tablet but the effect thereof is not satisfying.

### SUMMARY OF THE INVENTION

The object of the present invention is to provide an oral particle including pseudoephedrine hydrochloride and cetirizine dihydrochloride, which can perform good absorption efficiency, and quick, stable and long-term medicinal effect.

Another object of the present invention is to provide an oral medicine including pseudoephedrine hydrochloride and cetirizine dihydrochloride, which can perform good absorption efficiency, and quick, stable and long-term medicinal effect.

Accordingly, the oral particle of the present invention primarily includes a nucleus, a pseudoephedrine-hydrochloride layer coated outside the nucleus, a release-control layer coated outside the pseudoephedrine-hydrochloride layer, and a cetirizine-dihydrochloride layer coated outside the release-control layer. The nucleus has a diameter ranging 25˜40 mesh. The pseudoephedrine-hydrochloride layer can be coated with a first coating solution composed of pseudoephedrine hydrochloride, a binder, a lubricant and pure water/alcohol. The release-control layer can be coated with a second coating solution composed of a matrix, talc powder and pure water. The cetirizine-dihydrochloride layer can be coated with a third coating solution composed of cetirizine dihydrochloride, a binder, a lubricant and pure water/alcohol.

In the present invention, the nucleus is preferably made from sugar which comprises 65˜95 wt. % of sucrose and pharmaceutical inert or neutral starch.

In the first coating solution, the pure water/alcohol for dissolving pseudoephedrine hydrochloride, the binder and the lubricant has a weight ratio ranging from 80/20 to 20/80, and preferably about 50/50; and the solid content ranges 10˜30 wt. %, and preferably 20 wt. %.

In the second coating solution, the matrix preferably includes either methyl acrylate or ethyl cellulose.

In the third coating solution, the pure water/alcohol for dissolving cetirizine dihydrochloride, the binder and the lubricant has a weight ratio ranging from 80/20 to 20/80, and preferably about 50/50, and the contents of cetirizine dihydrochloride, the binder and the lubricant range 20˜40 wt. %, 8˜20 wt. % and 30˜60 wt. %, respectively, and the solid content ranges 15˜35 wt. %, and preferably about 27 wt. %.

In the present invention, the alcohol is preferably ethanol; the lubricant includes preferably includes at least one of talc powder and stearic acid; and the binder preferably includes at least one of hydroxyl propyl methyl cellulose, hydroxyl propyl cellulose and povidone.

The oral medicine of the present invention primarily includes a plurality of, for example, hundreds of, the oral particles aforementioned in a capsule, so that each capsule may contain 120 mg of active pseudoephedrine hydrochloride and 5 mg of active cetirizine dihydrochloride. The capsule can be made from an animal or a plant.

By distributing pseudoephedrine hydrochloride and cetirizine dihydrochloride into the particles and controlling their dissolution rates with the release-control layer, good absorption efficiency, and quick, stable and long-term medicinal effect can be achieved.

### BRIEF DESCRIPTION OF THE DRAWINGS

FIG. 1 is an in-vitro dissolution profile showing the released content of cetirizine dihydrochloride of the present invention.

FIG. 2 is an in-vitro dissolution profile showing the released content of pseudoephedrine hydrochloride of the present invention.

FIG. 3 is an in-vitro dissolution profile showing the released content of cetirizine dihydrochloride of the market tablets Zytec®.

FIG. 4 is an in-vitro dissolution profile showing released content of pseudoephedrine hydrochloride of the market tablets Zytec®.

### DETAILED DESCRIPTION OF THE PREFERRED EMBODIMENTS

To more clearly explain features, objects and effects, the present invention, preferred embodiments are described accompanied with figures.

In the present invention, the oral particle including pseudoephedrine hydrochloride and cetirizine dihydrochloride includes an inner core capable of controlling and releasing pseudoephedrine hydrochloride and an outer layer.

The inner core includes:
(a) a nucleus;
(b) a pseudoephedrine-hydrochloride layer coated outside the nucleus with a first coating solution composed of pseudoephedrine hydrochloride, a binder, a lubricant and pure water/alcohol;
(c) a release-control layer coated outside the pseudoephedrine-hydrochloride layer with a second coating solution composed of a matrix selected from either methyl acrylate or ethyl cellulose, talc powder and pure water.

The outer layer coated outside the release-control layer is substantially a cetirizine-dihydrochloride layer which is coated with a third coating solution composed of nonsedative antihistamine cetirizine dihydrochloride, a binder, a lubricant and pure water/alcohol.

Generally, components and contents of the oral particle are as follows:

### 1. Inner Core

### (a) Nucleus

The nucleus has a diameter ranging 25˜40 mesh and a weight percentage ranges 17˜27 wt. % on the basis of the oral particle, and preferably about 20 wt. %. The nucleus is preferably made from sugar composed of 65˜95 wt. % of sucrose and pharmaceutical inert or neutral starch.

### (b) Pseudoephedrine-Hydrochloride Layer

The pseudoephedrine-hydrochloride layer has a weight percentage ranges 40˜65 wt. % on the basis of the oral particle, and preferably 54 wt. %. The components of the first coating solution include pseudoephedrine hydrochloride, binder, lubricant and pure water/alcohol for dissolving the other components. The weight ratio of pure water/alcohol ranges 80/20˜20/80, and preferably about 50/50. the solid content ranges 10˜30 wt. % on the basis of the third coating solution, and preferably 20 wt. %. The components of the lubricant include talc powder and stearic acid; and the components of the binder include hydroxyl propyl methyl cellulose, hydroxyl propyl cellulose and povidone.

### (c) Release-Control Layer

The release-control layer has a weight percentage ranges 15˜30 wt. % on the basis of the oral particle, and preferably 26 wt. %. The release-control layer includes 40˜60 wt. % of methyl acrylate, 5˜10 wt. % of talc powder and 35˜50 wt. % of pure water for diluting the other components. Preferably, the contents of methyl acrylate, the talc powder and pure water are 50 wt. %, 7 wt. % and 43 wt. %, respectively.

### 2. Outer Layer

The tablet film, substantially a cetirizine-dihydrochloride layer, has a weight percentage ranges 3˜8 wt. % on the basis of the oral particle, and preferably 4.5 wt. %. by dissolving proper amounts of cetirizine dihydrochloride, the binder and the lubricant in the solvent including pure water and alcohol, the third coating solution is formed. In the third coating solution, contents of cetirizine dihydrochloride, the binder and the lubricant are 20˜40 wt. %, 8˜20 wt. % and 30˜60 wt. %, respectively. The weight ratio of pure water/alcohol ranges 80/20˜20/80, and preferably 50/50. The solid content of the third coating solution ranges 15˜35 wt. %, and preferably 27 wt. %.

Preferably, alcohol is ethanol.

A plurality of the particles can be packaged or processed as various medicines, for example, a capsule containing hundreds of the particles. The capsule can be made from an animal or a plant. Preferably, each capsule includes 120 mg of active pseudoephedrine hydrochloride and 5 mg of active cetirizine dihydrochloride.

The nucleus having a diameter about 25˜40 mesh is suitable for operation in a rotary-spray granulator. The spraying speed should be carefully adjusted to avoid drying of the coating solution at a low speed and congregation at a high speed. To overcome these problems, a lower initial speed can be applied and then the speed is increased when the particles are growing larger.

The air flowing rate can be controlled with an outlet valve and optimized according to circulation of the particles. A lower flowing rate will reduce amount of the pellets; and a higher rate may hinder the particles circulating. In the preferred embodiments, the valve is adjusted to about 50 wt. % of the maximum at beginning and then gradually to about 60 wt. %.

During the coating process, temperature of the introduced air is about 40° C.˜50° C., and rotary speed of the disk is about 60˜100 rpm.

After the coating process, a drying process for about 20˜30 minutes is necessary to remove additional solvents.

### Example 1

### (a) Preparing the First Coating Solution for the Pseudoephedrine-Hydrochloride Layer

In a container, pure water (250 kg) and ethanol (250 kg) are added and mixed. Then pseudoephedrine hydrochloride (92.4 kg), stearic acid (23.1 kg), talc powder (7.55 kg) and hydroxyl propyl cellulose (4.2 kg) are added into the container to obtain the first coating solution. The first coating solution is filtered with a sieve of 80-mesh.

### (b) Preparing the Second Coating Solution for the Control-Release Layer

Talc powder (12.4 kg) is mixed with pure water (60 kg) which is blended in a container. Then the mixture is filtered with a sieve of 100-mesh. In another container, methyl acrylate (66 kg) is added and blended with a mixer, then the filtered liquid is slowly added and mixed well to obtain the second coating solution. The second coating solution is filtered with a sieve of 100-mesh.

### (c) Preparing the Third Coating Solution for the Cetirizine-Dihydrochloride Layer

In a container, pure water (15.4 kg) and ethanol (15.4 kg) are blended, and hydroxyl propyl methyl cellulose (1540 g) is then added and completely dissolved. Then cetirizine dihydrochloride (3850 g) and talc powders (6160 g) are added and mixed to obtain the third coating solution. The third coating solution is filtered with a sieve of 100-mesh.

### (d) Loading the Pseudoephedrine-Hydrochloride Layer

In a rotating granulator equipped with a disk of 1 m diameter, sugar nuclei (47.6 kg, 25˜30 mesh or 590˜710 μm) are placed. Temperature and flowing rate of the inlet gas is about 45° C.-55° C. and about 30˜35 m³/min, flowing rate of the outlet gas is about 35˜38 m³/min, rotary speed of the disk is about 60˜80 rpm, pressure of the injector is about 5.0˜6.0 kg/cm², and the loading is gradually increased from 500 g/min to 800 g/min. After complete loading, the particles are dried with 50˜60° C. air for 20 minutes.

### (e) Coating the Control-Release Layer

The dried particles are continued coated. Temperature of the inlet gas is about 35˜40° C. Flowing rates of the inlet and outlet gas are remained. Rotary speed of the disk is about 80˜90 rpm. Pressure of the injector is about 6.0 kg/cm². The loading is about 500˜700 g/min.

### (f) Coating the Cetirizine-Dihydrochloride Layer

The particles are continued coated. Temperature of the inlet gas is about 45˜55° C. Flowing rates of the inlet and outlet gas are remained. Rotary speed of the disk is about 60˜80 rpm. Pressure of the injector is about 5.0 kg/cm². The loading is about 600˜800 g/min.

### (g) Drying

After complete the coating process and stop spraying, rotary speed of the disk is reduced to 30 rpm, temperature of the inlet gas is increased to 55˜60° C. for 30 minutes for drying the particles. The particles are then cooled to about 25° C. and discharged into a proper container.

### (h) Screening

The particles are screened with a multiple sieve of 14 mesh and 30 mesh. The particles are classified into “no good” (over 14 mesh), “good” (14˜30 mesh), and “no good” (below 30 mesh).

### (i) Encapsulating

The “good” particles are encapsulated into No. 0 capsules by an automatic machine. Each capsule contains about 334 mg of particles, i.e., about 5 mg of cetirizine dihydrochloride and 120 mg of pseudoephedrine hydrochloride.

### Example 2

### (a) Loading the Pseudoephedrine-Hydrochloride Layer

Four equivalent amounts of binder solutions are prepared by adding hydroxyl propyl cellulose into ethanol which is blended in a container. Total amount of ethanol is 26250 g, and total amount of hydroxyl propyl cellulose is 1050 g.

Then pseudoephedrine hydrochloride (92.4 kg), stearic acid (23.1 kg) and talc powder (7.55 kg) are crushed and separated into four equivalent amounts of powders.

One selected from four equivalent amounts of sugar nuclei (47.6 kg, 25˜30 mesh or 590˜710 μm) is placed in a centrifugal granulating coater

(CF-1000) which is operated at 120˜150 rpm and 1000 NL/min˜1500 NL/min of gas flowing rate. An equivalent amount of the binder solution is slowly added at a rate of 250 g/min until 300 g. The powders are added for loading drugs until all powders are added and unloaded. The unloaded particles are then dried in a dryer at 50° C. for 4 hours.

### (b) Preparing the Second Coating Solution for the Control-Release Layer

Talc powder (12.4 kg) is mixed with pure water (60 kg) which is blended in a container. Then the mixture is filtered with a sieve of 100-mesh. In another container, methyl acrylate (66 kg) is added and blended with a mixer, then the filtered liquid is slowly added and mixed well to obtain the second coating solution. The second coating solution is filtered with a sieve of 100-mesh.

### (c) Preparing the Third Coating Solution for the Cetirizine-Dihydrochloride Layer

In a container, pure water (15.4 kg) and ethanol (15.4 kg) are blended, and hydroxyl propyl methyl cellulose (1540 g) is then add and completely dissolved. Then cetirizine dihydrochloride (3850 g) and talc powders (6160 g) are added and mixed to obtain the third coating solution. The third coating solution is filtered with a sieve of 100-mesh.

### (d) Coating the Control-Release Layer

In a rotating granulator equipped with a disk of lm diameter, the dried particles are continued coated. Temperature of the inlet gas is about 35˜40° C. Flowing rates of the inlet and outlet gas are 30˜35 m³/min and 35˜38 m³/min, respectively. Rotary speed of the disk is about 80˜90 rpm. Pressure of the injector is about 6.0 kg/cm². The loading is about 500˜700 g/min.

### (e) Coating the Cetirizine-Dihydrochloride Layer

The particles are continued coated. Temperature of the inlet gas is about 45˜55° C. Flowing rates of the inlet and outlet gas are remained. Rotary speed of the disk is about 60˜80 rpm. Pressure of the injector is about 5.0 kg/cm². The loading is about 600˜800 g/min.

### (f) Drying

After complete the coating process and stop spraying, rotary speed of the disk is reduced to 30 rpm, temperature of the inlet gas is increased to 55˜60° C. for 30 minutes for drying the particles. The particles are then cooled to about 25° C. and discharged into a proper container.

### (g) Screening

The particles are screened with a multiple sieve of 14 mesh and 30 mesh. The particles are classified into “no good” (over 14 mesh), “good” (14˜30 mesh), and “no good” (below 30 mesh).

### (h) Encapsulating

The “good” particles are encapsulated into No. 0 capsules by an automatic machine. Each capsule contains about 334 mg of particles, i.e., about 5 mg of cetirizine dihydrochloride and 120 mg of pseudoephedrine hydrochloride.

According to the present invention, the particles including pseudoephedrine hydrochloride and cetirizine dihydrochloride can promote bioavailability as the solubility of cetirizine dihydrochloride is increased and pseudoephedrine hydrochloride is stable and can be released for a long time.

### Clinic Experiments:

The average excretion half time of cetirizine dihydrochloride in human body is 7.56±1.00 hours, and that of pseudoephedrine hydrochloride is 4.76±0.44 hours. The capsule including 334 mg of the particles of the present invention, i.e., 5 mg of cetirizine dihydrochloride and 120 mg of pseudoephedrine hydrochloride is compared with the market tablet Zytec® each including 5 mg of cetirizine dihydrochloride and 120 mg of pseudoephedrine hydrochloride.

The capsule medicine of the present invention and the market tablets (Zytec®) were taken for further comparison in two groups, respectively. The first groups (Group A: A1, A2, . . . , A6) of the present invention and market tablets (Zytec®) are analyzed for contents of pseudoephedrine hydrochloride and sampled after 15, 30, 45, 60, 90, 120, 180, 240, 360, 480, 600 and 720 minutes; the second groups (Group B: B1, B2, . . . , B6) of the present invention and market tablets (Zytec®) are analyzed for contents of cetirizine dihydrochloride and sampled after 15, 30, 45 and 60 minutes. Compared with the difference therebetween, it will be obvious to know the variance for the capsule medicine of the present invention is smaller than that of the market tablets (Zytec®) to further prove that the present invention can perform better absorption efficiency and steadier long-term medicinal effect than the market tablets (Zytec®).

The results are listed in Tables 1˜4; wherein Tables 1 and 3 respectively show data of pseudoephedrine hydrochloride from Group A, and Tables 2 and 4 respectively show data of cetirizine dihydrochloride from Group B. It's obvious that relative standard derivations (RSD) and variances of the drug released contents from the capsule medicine of the present invention are less than the market tablets (Zytec®), no matter cetirizine dihydrochloride or pseudoephedrine hydrochloride.

TABLE 1

| | Content of pseudoephedrine | | | | | | | |
|---|---|---|---|---|---|---|---|---|
| Time | hydrochloride from Group A (%) | | | | | | RSD | |
| (min.) | A1 | A2 | A3 | A4 | A5 | A6 | (%) | variance |
| 0 | 0 | 0 | 0 | 0 | 0 | 0 | 0.00 | 0.00 |
| 15 | 33.1 | 31.39 | 33.91 | 32.42 | 33.16 | 33.39 | 2.68 | 0.78 |
| 30 | 33.92 | 32.39 | 36.04 | 32.76 | 33.31 | 33.73 | 3.82 | 1.66 |
| 45 | 34.17 | 34.19 | 36.18 | 34.42 | 35.22 | 35.27 | 2.27 | 0.63 |
| 60 | 37.37 | 35.72 | 36.81 | 35.55 | 39.01 | 37.54 | 3.47 | 1.65 |
| 90 | 39.11 | 39.83 | 37.06 | 35.97 | 39.48 | 38.77 | 3.96 | 2.31 |
| 120 | 44.74 | 43.61 | 41.71 | 44.93 | 45.24 | 42.27 | 3.38 | 2.19 |
| 180 | 58.62 | 62.45 | 56.04 | 58.44 | 61.12 | 55.75 | 4.55 | 7.15 |
| 240 | 72.95 | 71.63 | 65.99 | 66.17 | 68.36 | 70.94 | 4.23 | 8.61 |
| 360 | 83.4 | 82.68 | 82.99 | 86.28 | 87.3 | 85.56 | 2.28 | 3.74 |
| 480 | 98.82 | 96.48 | 97.04 | 94.08 | 96.51 | 97.21 | 1.59 | 2.36 |
| 600 | 99.86 | 100.99 | 94.99 | 97.55 | 98.82 | 95.96 | 2.35 | 5.30 |
| 720 | 103.34 | 100.54 | 100.95 | 101.93 | 102.52 | 96.16 | 2.52 | 6.45 |

TABLE 2

| | Content of cetirizine dihydrochloride | | | | | | | |
|---|---|---|---|---|---|---|---|---|
| Time | from Group B (%) | | | | | | RSD | |
| (min.) | B1 | B2 | B3 | B4 | B5 | B6 | (%) | variance |
| 0 | 0 | 0 | 0 | 0 | 0 | 0 | 0.00 | 0.00 |
| 15 | 99.80 | 96.59 | 96.45 | 98.43 | 101.30 | 99.76 | 1.96 | 3.75 |
| 30 | 100.73 | 99.17 | 99.75 | 100.21 | 104.65 | 102.61 | 2.04 | 4.26 |
| 45 | 101.11 | 99.67 | 100.44 | 100.34 | 104.49 | 103.35 | 1.88 | 3.66 |
| 60 | 101.80 | 102.00 | 101.31 | 101.18 | 105.10 | 103.93 | 1.55 | 2.54 |
| 90 | 102.76 | 102.59 | 101.66 | 102.10 | 105.62 | 104.35 | 1.46 | 2.26 |

TABLE 4

| | Content of pseudoephedrine | | | | | | | |
|---|---|---|---|---|---|---|---|---|
| Time | hydrochloride from Group A (%) | | | | | | RSD | |
| (min.) | A1 | A2 | A3 | A4 | A5 | A6 | (%) | variance |
| 0 | 0 | 0 | 0 | 0 | 0 | 0 | 0 | 0 |
| 15 | 18.89 | 17.15 | 18.01 | 19.38 | 19.99 | 25.41 | 14.76 | 8.55 |
| 30 | 27.87 | 28.39 | 27.97 | 26.89 | 29.58 | 40.96 | 17.52 | 28.15 |
| 45 | 36.62 | 32.61 | 33.46 | 31.32 | 35.60 | 44.82 | 13.58 | 23.55 |
| 60 | 44.61 | 36.93 | 37.90 | 36.06 | 39.56 | 49.70 | 13.02 | 28.22 |
| 90 | 53.81 | 45.16 | 46.44 | 45.03 | 48.19 | 58.68 | 11.15 | 30.55 |
| 120 | 61.25 | 50.73 | 53.40 | 50.89 | 54.20 | 65.13 | 10.57 | 34.96 |
| 180 | 72.78 | 61.58 | 64.81 | 62.17 | 64.01 | 76.20 | 9.08 | 36.89 |
| 240 | 80.87 | 69.57 | 72.76 | 68.98 | 75.06 | 81.88 | 7.38 | 30.48 |
| 360 | 94.25 | 81.55 | 84.94 | 87.11 | 87.03 | 95.13 | 6.03% | 28.39 |
| 480 | 101.81 | 90.01 | 92.25 | 92.37 | 94.89 | 105.85 | 6.49% | 38.98 |
| 600 | 104.73 | 93.22 | 94.4 | 97.53 | 98.13 | 108.91 | 6.15% | 37.42 |
| 720 | 107.26 | 96.16 | 98.91 | 102.02 | 101.18 | 115.63 | 6.74% | 48.76 |

| | Content of cetirizine dihydrochloride | | | | | | | |
|---|---|---|---|---|---|---|---|---|
| Time | from Group B (%) | | | | | | RSD | |
| (min.) | B1 | B2 | B3 | B4 | B5 | B6 | (%) | variance |
| 0 | 0 | 0 | 0 | 0 | 0 | 0 | 0 | 0 |
| 15 | 86.82 | 62.39 | 81.35 | 61.72 | 90.75 | 70.89 | 16.49 | 155.56 |
| 30 | 90.03 | 80.11 | 83.67 | 73.53 | 94.27 | 86.00 | 8.66 | 53.70 |
| 45 | 93.71 | 83.85 | 87.82 | 80.61 | 96.33 | 88.82 | 6.64 | 34.58 |
| 60 | 98.85 | 85.44 | 88.27 | 80.96 | 97.35 | 90.66 | 7.64 | 47.58 |

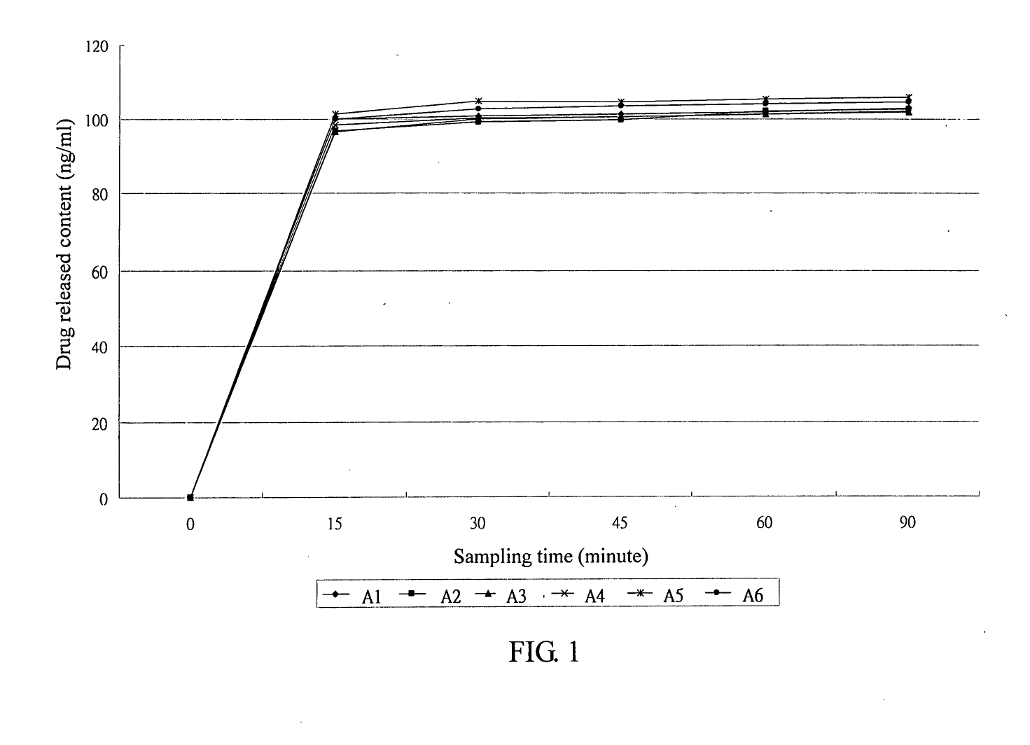

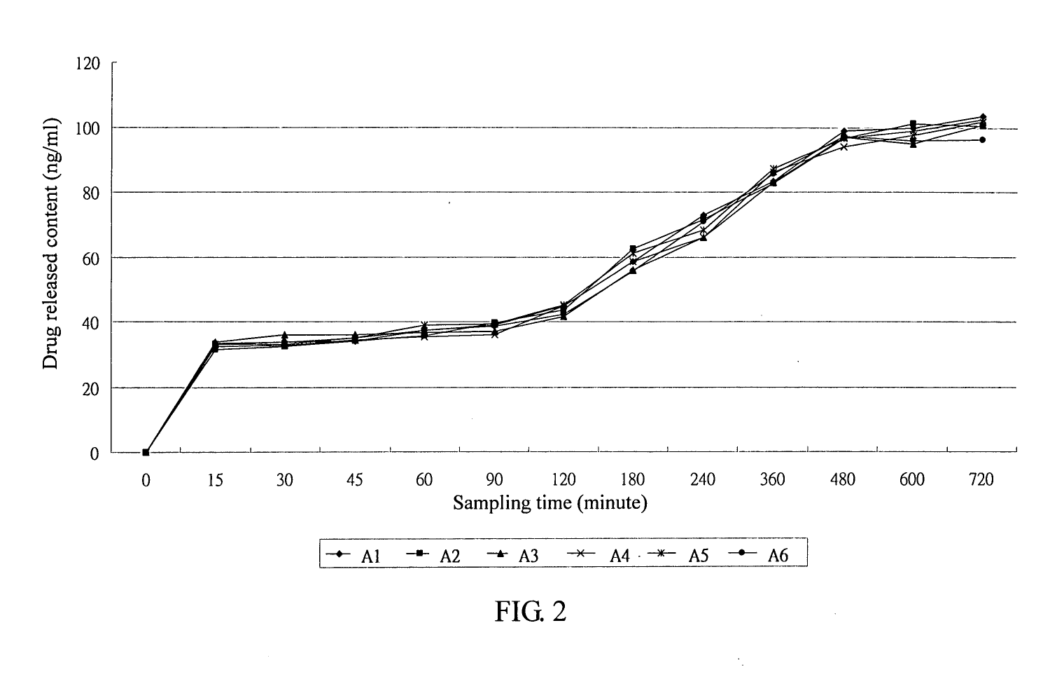

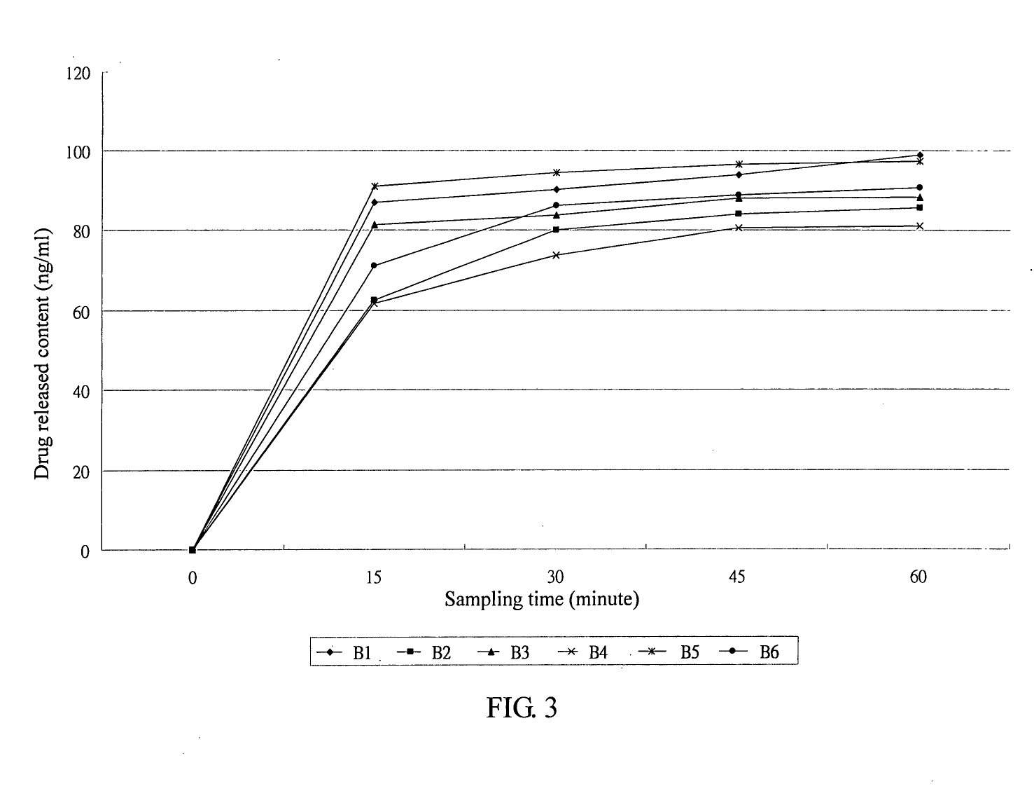

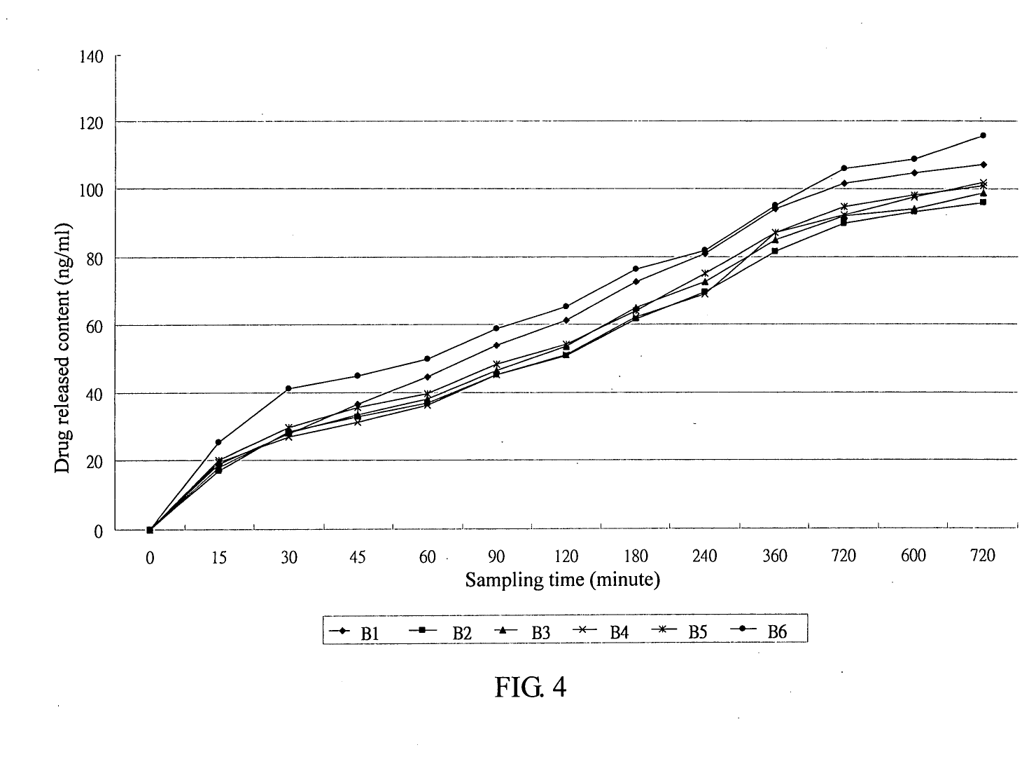

FIGS. 1˜4 also shows the in-vitro dissolution curves of released components, wherein FIG. 1 shows the released contents of cetirizine dihydrochloride from the Group B of the present invention, FIG. 2 shows the released contents of pseudoephedrine hydrochloride from Group A of the present invention, FIG. 3 shows the released contents of cetirizine dihydrochloride from Group B of the market tablets (Zytec®), and FIG. 4 shows the released contents of pseudoephedrine hydrochloride from Group A of the market tablets (Zytec®). It's also obvious that the particles taken by Group A and B of the present invention show better stability and longer releasing time than the market tablets (Zytec®) taken by Group A and B thereof.

According to the above, by distributing pseudoephedrine hydrochloride and cetirizine dihydrochloride into hundreds of the particles and controlling the dissolution rate with the release-control layer, the particles can perform good absorption efficiency, and quick, stable and long-term medicinal effect.

What is claimed is:

1. An oral particle including pseudoephedrine hydrochloride and cetirizine dihydrochloride, at least comprising an inner core capable of controlling and releasing pseudoephedrine hydrochloride and an outer layer; wherein
the inner core comprises:
(a) a nucleus having a diameter ranging about 25˜40 mesh;
(b) a pseudoephedrine-hydrochloride layer coated outside the nucleus with a first coating solution composed of pseudoephedrine hydrochloride, a binder, a lubricant and pure water/alcohol;
(c) a release-control layer coated outside the pseudoephedrine-hydrochloride layer with a second coating solution composed of talc powder, pure water and ethyl cellulose matrix; and
the outer layer is substantially a cetirizine-dihydrochloride layer which is coated outside the release-control layer with a third coating solution composed of nonsedative antihistamine cetirizine dihydrochloride, a binder, a lubricant and pure water/alcohol.

2. The oral particle as claimed in claim 1, wherein the nucleus is made from sugar which comprises 65˜95 wt. % of sucrose and pharmacuetical inert or neutral starch.

3. The oral particle as claimed in claim 1, wherein the weight ratio of the pure water/alcohol in the first coating solution ranges from 80/20 to 20/80.

4. The oral particle including pseudoephedrine hydrochloride and cetirizine dihydrochloride of claim 3, wherein the weight ratio of the pure water/alcohol in the first coating solution is about 50/50.

5. The oral particle as claimed in claim 1, wherein the alcohol is ethanol.

6. The oral particle as claimed in claim 1, wherein the solid content of pseudoephedrine hydrochloride, the binder and the lubricant in the first coating solution ranges 10˜30 wt. %.

7. The oral particle including pseudoephedrine hydrochloride and cetirizine dihydrochloride of claim 6, wherein the solid content of pseudoephedrine hydrochloride, the binder and the lubricant in the first coating solution is about 20 wt. %.

8. The oral particle as claimed in claim 1, wherein the lubricant is talc powder.

9. The oral particle as claimed in claim 1, wherein the binder is povidone.

10. The oral particle as claimed in claim 1, wherein the second coating solution comprises 40˜60 wt. % of methyl acrylate and 5˜10 wt. % of talc powder which both are diluted in 35˜50 wt. % of pure water.

11. The oral particle as claimed in claim 10, wherein the second coating solution comprises about 50 wt. % of methyl acrylate and about 7 wt. % of talc powder which both are diluted in about 43 wt. % of pure water.

12. The oral particle as claimed in claim 1, wherein the contents of cetirizine dihydrochloride, the binder and the lubricant in the third coating solution range 20˜40 wt. %, 8˜20 wt. % and 30˜60 wt. %, respectively.

13. The oral particle as claimed in claim 1, wherein the weight ratio of the pure water/alcohol in the third coating solution ranges 80/20˜20/80.

14. The oral particle as claimed in claim 13, wherein the weight ratio of the pure water/alcohol in the third coating solution is about 50/50.

15. The oral particle as claimed in claim 1, wherein the solid content of cetirizine dihydrochloride, the binder, the lubricant and the pure water/alcohol in the third coating solution ranges 15˜35 wt. %.

16. The oral particle as claimed in claim 15, wherein the solid content of cetirizine dihydrochloride, the binder, the lubricant and the pure water/alcohol in the third coating solution is about 27 wt. %.

17. An oral medicine including pseudoephedrine hydrochloride and cetirizine dihydrochloride, comprising:
a plurality of the oral particles as claimed in claim 1; and
a capsule made from an animal or a plant for packing the oral particles therein so that the oral medicine contains 120 mg of active pseudoephedrine hydrochloride and 5 mg of active cetirizine dihydrochloride.

18. The oral particle as claimed in claim 1, which comprises 17˜27 wt. % of the nucleus, 40˜65 wt. % of the pseudoephedrine-hydrochloride layer, 15˜30 wt. % of the release-control layer and 3˜8 wt. % of the outer layer, all based on the oral particle.

19. The oral particle as claimed in claim 18, which comprises about 20 wt. % of the nucleus, about 54 wt. % of the pseudoephedrine-hydrochloride layer, 26 wt. % of the release-control layer and about 4.5 wt. % of the outer layer, all based on the oral particle.
